(19) United States
(12) Patent Application Publication
(10) Pub. No.: US 2024/0010591 A1
(43) Pub. Date: Jan. 11, 2024

(54) INTERGRATED PROCESS TO PRODUCE ALDEHYDES FROM METHANOL

(71) Applicant: Rohm and Haas Company, Collegeville, PA (US)

(72) Inventors: Joseph F. Dewilde, King of Prussia, PA (US); Kirk W. Limbach, Dresher, PA (US); Reetam Chakrabarti, Phoenixville, PA (US)

(21) Appl. No.: 18/251,627

(22) PCT Filed: Nov. 17, 2021
(86) PCT No.: PCT/US2021/059593

§ 371 Date: May 3, 2023

Related U.S. Application Data
(63) Continuation of application No. 63/119,734, filed on Dec. 1, 2020.

(51) Int. Cl. C07C 45/50; C07C 45/78; C07C 67/44; C07C 1/24

(52) U.S. Cl. CPC C07C 45/50; C07C 45/78; C07C 67/44; C07C 1/24

(57) ABSTRACT

A process for preparing aldehydes from methanol includes introducing a feed stream comprising methanol and hydrogen gas into a reaction zone of a first reactor, converting the feed stream into an intermediate stream comprising C₂ to C₄ olefins in the reaction zone in the presence of a first catalyst, wherein the first catalyst is a microporous catalyst component, removing water and species C₄ and heavier from the intermediate stream to form a lights stream, and converting the lights stream into a product stream comprising propionaldehyde in the presence of a second catalyst and carbon monoxide in a second reactor. The propionaldehyde can further be converted to methyl methacrylate via oxidative esterification.

### FIELD

The present specification generally relates to processes that efficiently convert a methanol-containing streams to aldehydes via C₂ to C₄ hydrocarbons.

### TECHNICAL BACKGROUND

For a number of industrial applications, hydrocarbons are used, or are starting materials used, to produce plastics, fuels, and various downstream chemicals. C₂ to C₄ hydrocarbons are particularly useful in downstream applications, such as, for example, preparing aldehydes and further products, such as methyl methacrylate (MMA). MMA is a high-value chemical intermediate for the production of (meth)acrylic polymers and copolymers.

A variety of processes for producing lower hydrocarbons has been developed, including petroleum cracking and various synthetic processes. However, such process typically require separation of several light species, which are costly and difficult to separate.

Other improvements have been made to the formation of olefins from methanol. For example, Arora et al., Nature Catalysis 1, 666-672 (2018), discloses that hydrogen can be co-fed to the methanol-to-olefin process to improve the lifetime of the catalyst. This process, however, is costly because the hydrogen is not used as a reactant in the methanol-to-olefin process and the hydrogen requires extra handling/separation and/or recycling to the reactor.

Accordingly, a need exists for processes and systems in which aldehydes and/or methyl methacrylate can be produced from methanol efficiently and with high yield.

### SUMMARY

One aspect of the present invention relates to a process comprising introducing a feed stream comprising methanol and hydrogen gas into a reaction zone of a first reactor, converting the feed stream into an intermediate stream comprising C₂ to C₄ olefins in the reaction zone in the presence of a first catalyst, wherein the first catalyst is a microporous catalyst, removing water and C₄ and higher hydrocarbons from the intermediate stream to form a lights stream, and converting the lights stream into a product stream comprising propionaldehyde and/or butyraldehyde in the presence of a second catalyst and carbon monoxide in a second reactor.

Additional features and advantages will be set forth in the detailed description which follows, and in part will be readily apparent to those skilled in the art from that description or recognized by practicing the embodiments described herein, including the detailed description which follows and the claims.

It is to be understood that both the foregoing general description and the following detailed description describe various embodiments and are intended to provide an overview or framework for understanding the nature and character of the claimed subject matter.

### DETAILED DESCRIPTION

Reference will now be made in detail to embodiments of processes utilizing methanol to prepare C₂ to C₄ olefins and further to aldehydes and/or methyl methacrylate.

In general, in methanol to hydrocarbon processes, costly separations are carried out to separate light species from the desired species, such as ethylene or propylene. However, it has been discovered that the light species can be passed through with the desired species to a second reactor to produce desired aldehydes, which may then be easily separated out for further processing, such as converting propionaldehyde to methyl methacrylate.

By eliminating the need for separating light species formed in the methanol-to-olefin reaction, significant savings can be achieved in capital and operating costs.

In the process of the present invention, a feed stream comprising methanol and hydrogen is introduced into a reaction zone of a first reactor. In the first reactor, the hydrogen does not react, but instead serves to improve the lifetime of the first catalyst.

The hydrogen may present in the feed stream in an amount of from 10.0 vol % to 90.0 vol % H2, such as from 30.0 vol % to 85.0 vol % H2, from 30.0 vol % to 80.0 vol % H2 based on the total volume of the feed stream. Preferably, the amount of hydrogen in the feed stream is at least 50 vol %, and more preferably at least 60 vol %, and even more preferably at least 70 vol % based on the total volume of the feed stream. The amount of hydrogen in the feed stream can be adjusted to achieve the desired improvements in the first catalyst lifetime.

The methanol may be present in the feed stream in an amount of from 3.0 vol % to 20.0 vol % methanol, such as, for example, from 5.0 vol % to 20.0 vol % methanol or from 10.0 vol % to 20.0 vol. %, relative to the total volume of the feed stream.

The first catalyst is a microporous catalyst, such as, for example, a zeolite.

The microporous catalyst component is preferably selected from molecular sieves having 8-MR pore openings and having a framework type selected from the group consisting of the following framework types CHA, AEI, AFX, ERI, LTA, UFI, RTH, EDI, GIS, MER, RHO, and combinations thereof, the framework types corresponding to the naming convention of the International Zeolite Association. It should be understood both aluminosilicate and silicoaluminophosphate frameworks may be used. The microporous catalyst component may include tetrahedral aluminosilicates, ALPOs (such as, for example, tetrahedral aluminophosphates), SAPOs (such as, for example, tetrahedral silicoaluminophosphates), and silica-only based tectosilicates. The microporous catalyst component may be silicoaluminophosphate having a Chabazite (CHA) framework type. Examples of these may include, but are not necessarily limited to: CHA framework types selected from SAPO-34 and SSZ-13; and AEI framework types such as SAPO-18. Combinations of microporous catalyst components having any of the above framework types may also be employed. It should be understood that the microporous catalyst component may have different membered ring pore opening depending on the desired product. For instance, microporous catalyst component having 8-MR to 12-MR pore openings could be used depending on the desired product.

The reaction conditions within the reaction zone of the first reactor will now be described. The feed stream contacted with the first catalyst in the reaction zone of the first reactor under reaction conditions sufficient to form an intermediate stream comprising C2 to C4 olefins. The intermediate stream may further comprise other hydrocarbons, e.g., C5 or higher hydrocarbons. Preferably, the intermediate stream comprises primarily C2 to C4 olefins. The reaction conditions comprise a temperature within the reaction zone ranging, for example, from 300° C. to 500° C., such as from 380° C. to 450° C., from 380° C. to 440° C., from 380° C. to 430° C., from 380° C. to 420° C., from 380° C. to 410° C., from 380° C. to 400° C., or from 380° C. to 390° C.

The reaction conditions also include, for example, a pressure inside the reaction zone of at least ambient pressure (1 bar or 100 kPa). To further improve the catalyst lifetime improvements due to the presence of hydrogen in the first reactor, higher pressures inside the reaction zone of the first reactor may also be used. For example, the pressure inside the reaction zone could be 5 bar (500 kPa), 10 bar (1,000 kPa), or higher.

The intermediate stream comprises C2 to C4 olefins and hydrogen, which passes unreacted through the first reactor, as well as hydrocarbons and paraffins.

Water is removed from the intermediate stream. Additionally C4 and higher hydrocarbons are also removed from the intermediate stream to form a lights stream, which is fed to the second reactor without further processing or separation. Depending on the desired aldehydes, C3 hydrocarbons can be removed with the C4 and higher hydrocarbons. Alternatively, the C3 hydrocarbons can be sent in the overhead stream as part of the lights stream for conversion to butyraldehyde in the hydroformylation or oxo process in the second reactor. Butyraldehyde can be used to make n-butanol or 2-ethylhexanol. Catalysts for this process include, but are not limited to, (organo)phosphines, phosphites, or bidentate ligand complexes comprising Group VIII and VIIIB metals.

The lights stream comprises ethylene and optionally the C3 hydrocarbons, including propylene, as well as hydrogen. The lights stream is converted into a product stream in the presence of carbon monoxide, which is added, and a second catalyst in a second reactor. The lights stream is subjected to a hydroformylation reaction or oxo process in the second reactor to form aldehydes from the olefins present in the second feed stream. If the C3 hydrocarbons are removed from the intermediate stream with the higher hydrocarbons, the primary product of the second reactor is propionaldehyde. When the C3 hydrocarbons are not removed from the intermediate stream, the product of the second reactor primarily comprises a mixture of propionaldehyde and butyraldehyde. Any paraffins present in the lights stream pass through the second reactor and can be recycled to the feed stream.

Advantageously, the hydrogen, which passes through the first reactor, is consumed in the oxo process. By reacting the hydrogen in the second reactor, the inventive process avoids costly handling/separation of the hydrogen from the other light gases. Therefore, the presence of hydrogen in the inventive process is at least two-fold. In the first reactor, the hydrogen improves the lifetime of the catalyst. In conventional processes, the hydrogen would then need to be separated and recycled to the first reactor. However, the inventive process, costly and cumbersome separation of hydrogen from the intermediate stream is avoided because the hydrogen passes through to the second reactor to be consumed in the oxo process. While unreacted hydrogen exiting the oxo process can be recycled, the amount of hydrogen that may need to be recycled is significantly reduced.

The aldehyde products, e.g., propionaldehyde and butyraldehyde, are preferably separated from the product stream. The separation of the aldehyde products from lighter gases in the product stream is much easier than the separation of ethylene and/or propylene from the intermediate stream, which makes the inventive process significantly more efficient than conventional processes.

Propionaldehyde from the product stream may be further used to form methyl methacrylate via an oxidative esterification reaction using any known method. For example, propionaldehyde can be converted to methacrolein in the presence of formaldehyde. The methacrolein can subsequently be converted to methyl methacrylate using any known catalyst.

It will be apparent to those skilled in the art that various modifications and variations can be made to the embodiments described herein without departing from the spirit and scope of the claimed subject matter. Thus, it is intended that the specification cover the modifications and variations of the various embodiments described herein provided such modification and variations come within the scope of the appended claims and their equivalents.

1. A process comprising:
introducing a feed stream comprising methanol and hydrogen into a reaction zone of a first reactor;
converting the feed stream into an intermediate stream comprising C₂ to C₄ olefins in the reaction zone in the presence of a first catalyst, wherein the first catalyst microporous catalyst;
removing water and species C₄ and heavier from the intermediate stream to form a lights stream; and
converting the lights stream into a product stream comprising propionaldehyde in the presence of a second catalyst and carbon monoxide in a second reactor.

2. The process of claim 1, wherein the product stream further comprises butyraldehyde.

3. The process of claim 1, wherein the step of removing water and species C₄ and heavier comprises removing water and species C₃ and heavier to form the lights stream.

4. The process of claim 1, wherein the hydrogen present in the feed stream is passed with the lights stream to the second reactor.

5. The process of claim 1, further comprising removing the propionaldehyde from the product stream.

6. The process of claim 1, wherein the first reactor is operated at a pressure greater than atmospheric pressure.

7. The process of claim 1, wherein the hydrogen is present in the feed stream in an amount of at least 50 vol % based on the total volume of the feed stream.

8. The process of claim 1, wherein the microporous catalyst component is a molecular sieve having 8-MR pore openings.

9. The process of claim 1, further comprising converting the propionaldehyde to methyl methacrylate via oxidative esterification.
